(19) United States
(12) Patent Application Publication
(10) Pub. No.: US 2011/0276007 A1
(43) Pub. Date: Nov. 10, 2011

(54) SAFETY NEEDLE DEVICE

(76) Inventor: Igor Denenburg, Oceanside, NY (US)

(21) Appl. No.: 13/062,073

(22) PCT Filed: Sep. 11, 2008
(86) PCT No.: PCT/US08/75952

§ 371 Date: Mar. 3, 2011

(51) Int. Cl. A61M 5/32

(52) U.S. Cl. 604/198

(57) ABSTRACT

A safety needle device comprising a needle cannula, a hub including locking element, a compression spring and a needle guard including a tip portion opening along the injection axis for the needle cannula, wall portion and resilient locking portion, which is resilient radially and perpendicularly with respect to the injection axis, the resilient locking portion being operative to move rearwardly with respect to the locking element along a first surface of the locking element during injection, against the urging of the spring, and to move forwardly with respect to the locking element along a second surface of the locking element following injection, under the urging of the spring, into locking engagement with the locking element preventing further movement of the needle guard along the injection axis, the first and second surfaces being mutually spaced from each other along an axis extending radially and perpendicularly with respect to the injection axis.

### REFERENCE TO RELATED APPLICATIONS

Reference is hereby made to U.S. Provisional Patent Application Ser. No. 60/973,179, filed Sep. 18, 2007, entitled SAFETY NEEDLE ASSEMBLY, the disclosure of which is hereby incorporated by reference and convention priority of which is hereby claimed pursuant to 37 CFR 1.78a (4) and (5)(i).

### FIELD OF THE INVENTION

The invention pertains to safety needle devices generally.

### BACKGROUND OF THE INVENTION

The following patent documents are believed to represent the current state of the art:

U.S. Pat. No. 4,813,940; U.S. Pat. No. 5,104,384; U.S. Pat. No. 5,389,085; U.S. Pat. No. 5,688,241; U.S. Pat. No. 6,855,129; US2005/0113750; US2006/0167411 and US2008/0167624.

### SUMMARY OF THE INVENTION

The present invention seeks to provide an improved safety needle device.

There is thus provided in accordance with a preferred embodiment of the present invention a safety needle device comprising a needle cannula, an integrally formed one-piece hub including a syringe attachment portion having a fluid flow channel, a needle cannula attachment portion for fixed attachment of the needle cannula thereto in fluid communication with the fluid flow channel along an injection axis and at least one locking element, the integrally formed one-piece hub defining a forward-facing spring seat, a compression spring arranged along the injection axis and seated on the forward-facing spring seat,

an integrally formed, one-piece needle guard including a tip portion having a forward facing injection site engagement surface, a rearward-facing spring seat defining surface and a throughgoing opening along the injection axis for the needle cannula, at least one side wall portion extending parallel to the injection axis and at least one resilient locking portion, which is resilient radially and perpendicularly with respect to the injection axis, the resilient locking portion being operative to move rearwardly with respect to at least one the locking element along a first surface of the at least one locking element during injection, against the urging of the compression spring, and to move forwardly with respect to the at least one locking element along a second surface of the at least one locking element following injection, under the urging of the compression spring, into locking engagement with said locking element preventing further movement of the needle guard along the injection axis, the first and second surfaces being mutually spaced from each other along an axis extending radially and perpendicularly with respect to the injection axis.

In accordance with a preferred embodiment of the present invention safety needle device, the locking element extends transversely to the injection axis.

In accordance with a preferred embodiment of the present invention safety needle device, the spring seat extends circumferentially of the needle cannula attachment portion.

In accordance with a preferred embodiment of the present invention safety needle device the first surface is located radially inward with respect to the second surface.

In accordance with a preferred embodiment of the present invention safety needle device the first surface is located radially inward with respect to the second surface.

### BRIEF DESCRIPTION OF THE DRAWINGS

The present invention will be understood and appreciated more fully from the following detailed description:

FIG. 1 is a simplified perspective assembled view illustration of the safety needle device constructed and operative in accordance with a preferred embodiment of the present invention;

FIGS. 2A, 2B and 2C are simplified pictorial illustrations of a needle cannula useful in the apparatus of FIG. 1;

FIGS. 3A and 3B are sectional illustrations taken along lines IVA-IVA and IVB-IVB in FIGS. 2B and 2C;

FIGS. 4A, 4B and 4C are simplified pictorial illustrations of a needle guard useful in the apparatus of FIG. 1;

FIGS. 5A and 5B are sectional illustrations taken along lines VIA-VIA and VIB-VIB in FIGS. 4B and 4C;

FIGS. 6A, 6B, 6C, 6D, 6E, 6F, 6G & 6H are simplified perspective view illustrations indicating various stages in the operation of the safety needle device of FIGS. 1-5B;

FIGS. 7A, 7B, 7C, 7D, 7E, 7F, 7G & 7H are sectional illustrations indicating various stages in the operation of the safety needle device of FIGS. 6A-6H.

### DETAILED DESCRIPTION OF THE DRAWINGS

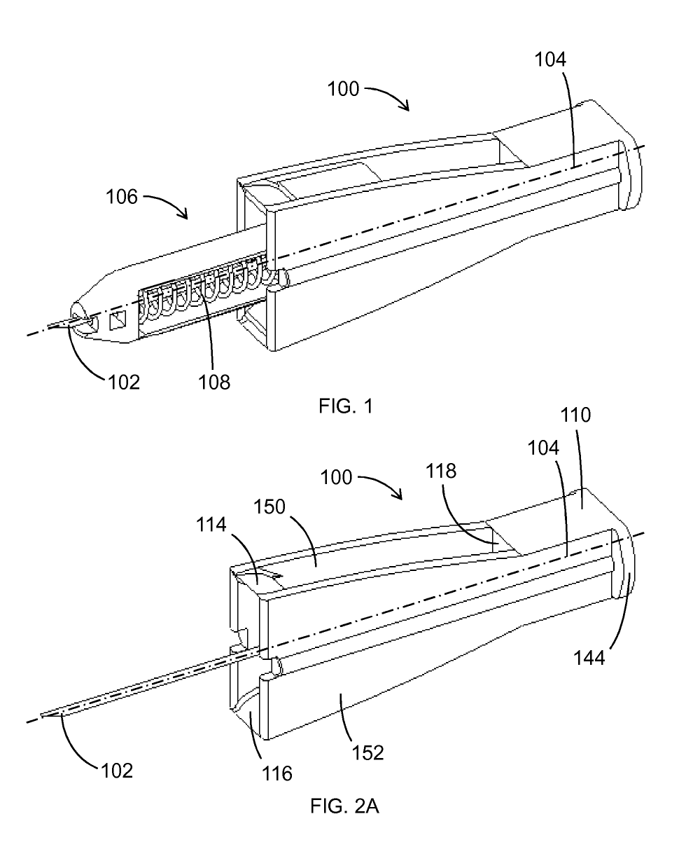

Reference is now made to FIGS. 1-6H which illustrate the structure and operation of the safety needle device constructed and operative in accordance with a preferred embodiment of the present invention.

FIG. 1 is a simplified perspective assembled view illustration of safety needle device constructed and operative in accordance with a preferred embodiment of the present invention. As seen in FIG. 1, the safety needle device includes a hub 100 which the needle cannula 102 extending along an injection axis 104 is fixed, typically by an adhesive, a needle guard 106 and spring 108.

In accordance with a preferred embodiment of the present invention, integrally formed one-piece hub 100 is configured at a syringe attachment portion 110 having a fluid flow channel 112 thereof to receive a needleless syringe (not shown) and needle cannula attachment portion 113 for fixed attachment of needle cannula 102 in fluid communication with fluid flow channel 112 along an injection axis 104 and at locking elements 114 and 116, the integrally formed one-piece hub defining a forward-facing spring seat 118.

The safety needle device also preferably includes a compression spring 108 arranged along injection axis 104 and seated on forward-facing spring seat 118.

In accordance with a preferred embodiment of the present invention, an integrally formed, one-piece needle guard 106 including a tip portion 120 having a forward facing injection site engagement surface 122, a rearward-facing spring seat defining surface 124 and a throughgoing opening along injection axis 104 for needle cannula 102, at wall portions 126 and 128 extending parallel to injection axis 104 and at resilient locking portions 130 and 132, which is resilient radially and perpendicularly with respect to injection axis 104, resilient locking portions 130 and 132 being operative to move rearwardly with respect to at locking elements 114 and 116 along first surfaces 134 and 136 of at locking elements 114 and 116 during injection, against the urging of compression spring 108, and to move forwardly with respect to at locking elements 114 and 116 along second surfaces 138 and 140 of locking elements 114 and 116 following injection, under the urging of compression spring 108, into locking engagement with locking elements 114 and 116 preventing further movement of needle guard 106 along injection axis 104, first and second surfaces being mutually spaced from each other along an axis 104 extending radially and perpendicularly with respect to injection axis 104.

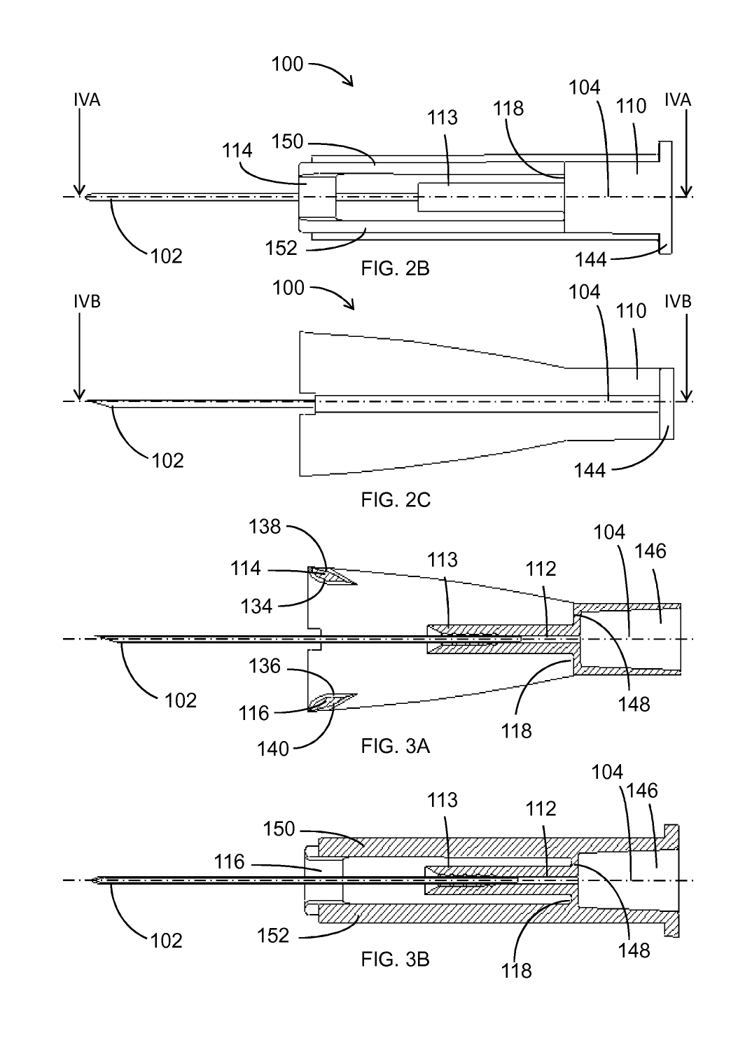

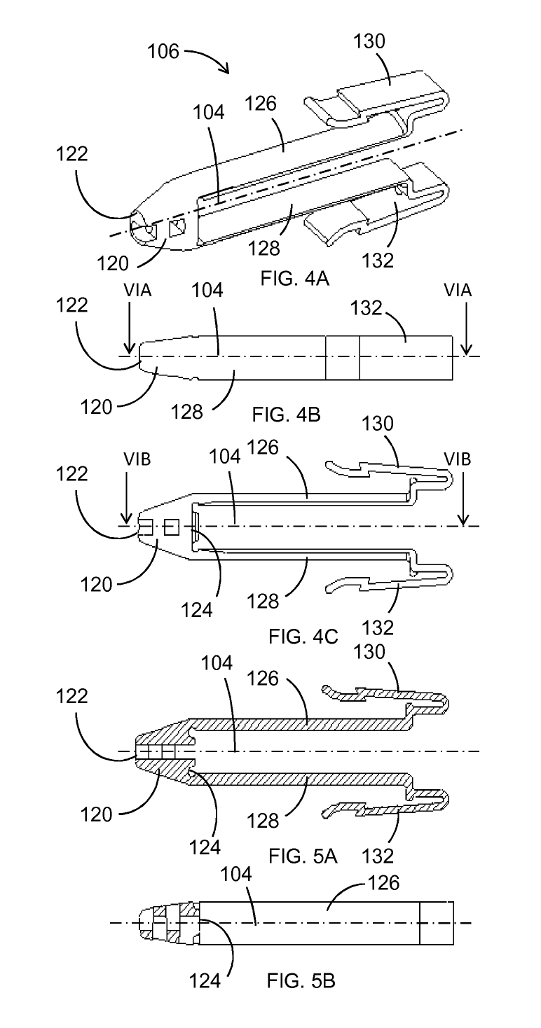

FIGS. 2, 2A, 2B, 3A and 3B, illustrate a preferred embodiment of the hub 100. As seen particularly in FIGS. 2-3B, the hub 100 is a generally cylindrical element defining a Luer lock at syringe attachment portion 110 and having an external threading 144 at syringe attachment portion 110 thereof and a generally conical internal passageway 146 opening at syringe attachment portion 110.

Passageway 146 terminates in a bulkhead 148 which defines fluid flow channel 112 in fluid communication with needle cannula 102.

First and second generally axially extending segments 150 and 152 are communicated at locking elements 114 and 116 with Luer lock at syringe attachment portion 110.

Locking element 114 has surfaces 134 and 138, and locking element 116 has surfaces 136 and 140.

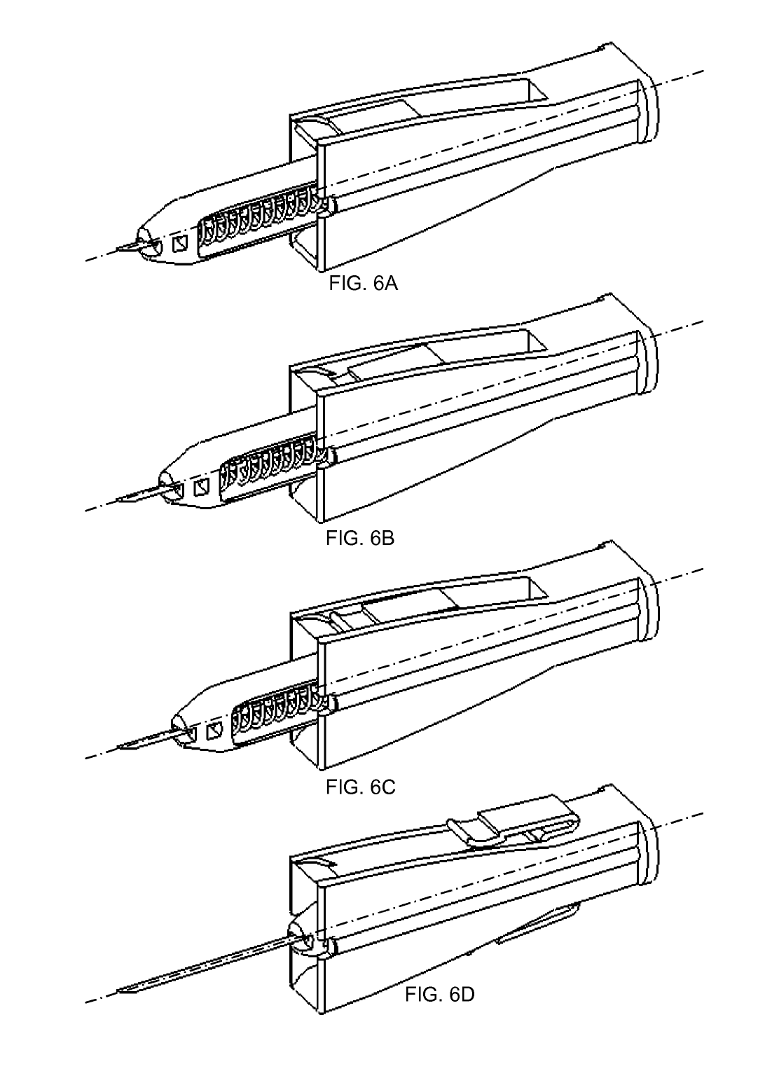

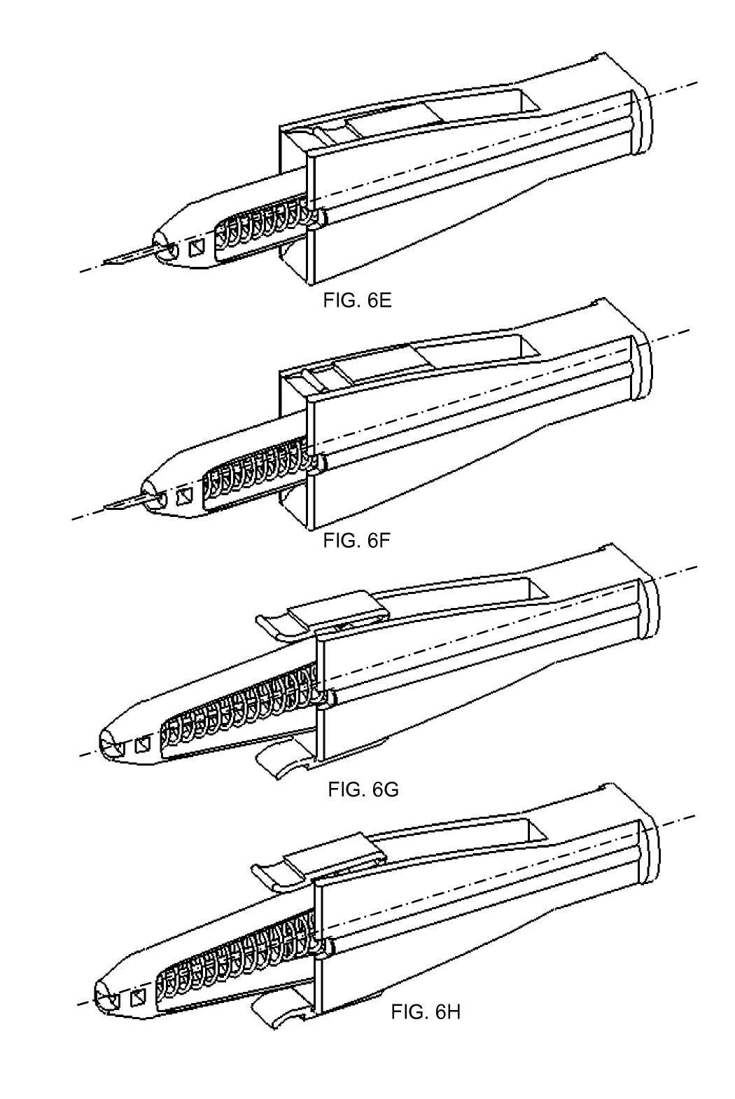

FIGS. 4, 4A, 4B, 5A and 5B illustrate a preferred embodiment of the needle guard 106. As seen in FIGS. 4-5B, the needle guard 106 is preferably a unitary element, typically injection molded and includes tip portion 120 having forward facing injection site engagement surface 122, a rearward-facing spring seat defining surface 124, wall portions 126 and 128 and resilient locking portions 130 and 132.

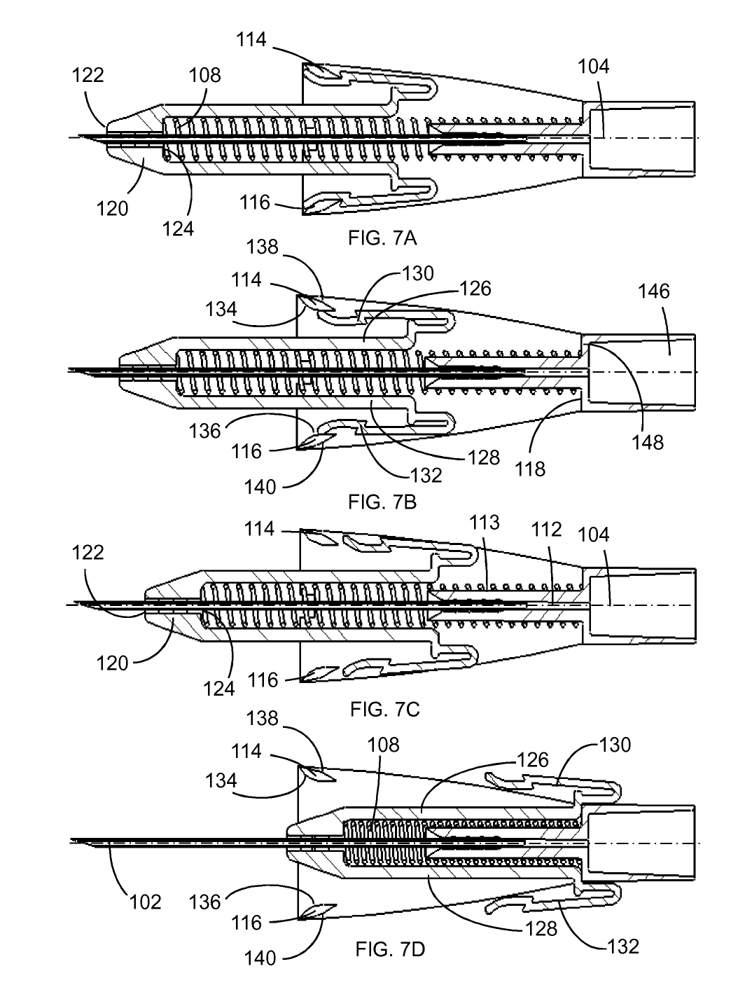

FIGS. 6A, 6B, 6C, 6D, 6E, 6F, 6G & 6H are simplified perspective view illustrations indicating various stages in the operation of the safety needle device of FIGS. 1-5B.

FIGS. 6A, 6B, 6C and 6D resilient locking portions 130 and 132 being operative to move rearwardly with respect to locking elements 114 and 116 along first surfaces 134 and 136 of locking elements 114 and 116 during injection, against the urging of compression spring 108.

FIGS. 6E, 6F, 6G and 6H resilient locking portions 130 and 132 being operative to move forwardly with respect to locking elements 114 and 116 along second surfaces 138 and 140 of locking elements 114 and 116 following injection, under the urging of compression spring 108, into locking engagement with locking elements 114 and 116 preventing further movement of needle guard 106 along injection axis 104.

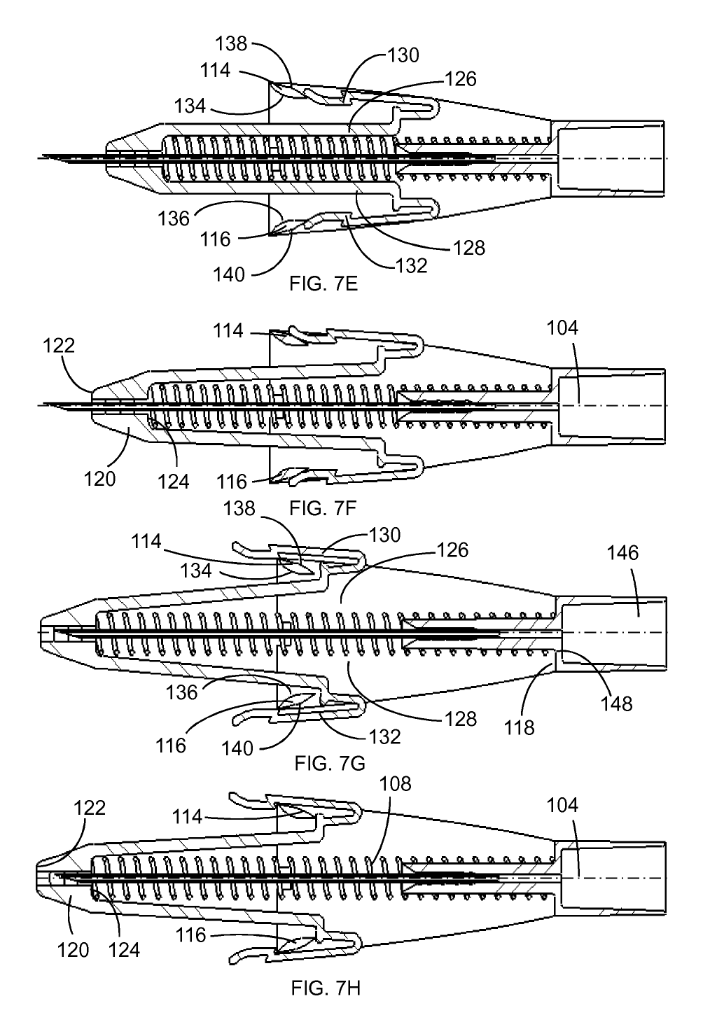

FIGS. 7A, 7B, 7C, 7D, 7E, 7F, 7G & 7H are sectional illustrations the safety needle device in FIGS. 6A-6H.

1. A safety needle device comprising:
a needle cannula;
an integrally formed one-piece hub including a syringe attachment portion having a fluid flow channel, a needle cannula attachment portion for fixed attachment of said needle cannula thereto in fluid communication with said fluid flow channel along an injection axis and at least one locking element, said integrally formed one-piece hub defining a forward-facing spring seat;
a compression spring arranged along said injection axis and seated on said forward-facing spring seat;
an integrally formed, one-piece needle guard including a tip portion having a forward facing injection site engagement surface, a rearward-facing spring seat defining surface and a throughgoing opening along said injection axis for said needle cannula, at least one side wall portion extending parallel to said injection axis and at least one resilient locking portion, which is resilient radially and perpendicularly with respect to said injection axis, said resilient locking portion being operative to move rearwardly with respect to at least one said locking element along a first surface of said at least one locking element during injection, against the urging of said compression spring, and to move forwardly with respect to said at least one locking element along a second surface of said at least one locking element following injection, under the urging of said compression spring, into locking engagement with said locking element preventing further movement of said needle guard along said injection axis, said first and second surfaces being mutually spaced from each other along an axis extending radially and perpendicularly with respect to said injection axis.

2. A safety needle device according to claim 1 and wherein said locking element extends transversely to said injection axis.

3. A safety needle device according to claim 1 and wherein said spring seat extends circumferentially of said needle cannula attachment portion.

4. A safety needle device according to claim 2 and wherein said spring seat extends circumferentially of said needle cannula attachment portion.

5. A safety needle device according to claim 1 and wherein said first surface is located radially inward with respect to said second surface.

6. A safety needle device according to claim 2 and wherein said first surface is located radially inward with respect to said second surface.

7. A safety needle device according to claim 3 and wherein said first surface is located radially inward with respect to said second surface.

8. A safety needle device according to claim 4 and wherein said first surface is located radially inward with respect to said second surface.
